(19) United States
(12) Patent Application Publication
(10) Pub. No.: US 2022/0379046 A1
(43) Pub. Date: Dec. 1, 2022

(54) Device For Assisting With The Use Of A Device For Dispensing A Product

(71) Applicant: Nemera La Verpillière, LA VERPILLIERE (FR)

(72) Inventors: Thierry Decock, Lyon (FR); Joris Pinto, Saint-Priest (FR); Lenaic Guimard, Saint-Cassien (FR); Gaëtan Painchaud, Francheville (FR); Olivier Vernant, Anthon (FR)

(21) Appl. No.: 17/773,797

(22) PCT Filed: Oct. 30, 2020
(86) PCT No.: PCT/EP2020/080557

§ 371 Date: May 2, 2022

(30) Foreign Application Priority Data
Oct. 31, 2019 (FR) FRI912304

(51) Int. Cl. A61M 11/00; A61M 15/08; B05B 11/00

(52) U.S. Cl. CPC A61M 11/006; A61M 15/08; B05B 11/3059; A61M 2205/276

(57) ABSTRACT

An assistance device for a product dispensing device is provided, in which product dispensing is implemented by displacement or deformation of a moving part of the dispensing device between a rest position and an activation position. The assistance device includes an engaging unit, a locking unit configured to prevent the dispensing of the product, an actuator configured to switch the locking unit from a locked position to an unlocked position, a return unit configured to switch the locking unit from the unlocked position to the locked position, out of the activation position of the product dispensing, a blocking unit for blocking the locking unit in the unlocked position, a first disengagement member for disengaging the blocking unit configured to be triggered upon the activation of the dispensing device, and a second disengagement member for disengaging the blocking unit configured to be triggered independently of the activation of the dispensing device.

### FIELD OF THE INVENTION

The invention relates to a device for assisting in the use of a product dispensing device.

### BACKGROUND OF THE INVENTION

The state of the art, in particular document US-A1-2010/084433 already describes a device for assisting in the use of a product dispensing device comprising locking means which prevent the product dispensing by the dispensing device by blocking the displacement of a moving part of a pump of the dispensing device to prevent an unauthorised person from using the dispensing device or to prevent the delivery of product during a given period of time. The assistance device comprises blocking means for blocking the locking means in the unlocked position. When the blocking means hold the locking means in the unlocked position, the only way to disengage the blocking means, and therefore return to the locked position, is to activate the device for dispensing a product and therefore deliver a dose of product. Thus, the dispensing device must be activated to allow the locking means to return to the locked position.

### SUMMARY OF THE INVENTION

Such a device has disadvantages. In particular, a dose of product is wasted when the user wants to relock the dispensing device but does not want to deliver a dose of product. Furthermore, it is not possible to provide for an automatic relocking that is independent from the dispensing of product.

The invention aims in particular to provide an assistance device for the use of a product dispensing device which overcomes at least some of these disadvantages.

Thus, the invention relates in particular to an assistance device for assisting in the use of a product dispensing device, the product dispensing being achieved by displacement or deformation of a moving part of the dispensing device between a rest position and an activation position, the assistance device comprising:
engaging means to the product dispensing device,
locking means capable of preventing the dispensing of the product,
an actuator configured to switch the locking means from a locked position in which they prevent the dispensing of the product to an unlocked position in which they do not prevent the dispensing of the product,
returns means configured to switch the locking means from the unlocked position to the locked position, out of the activation position of the product dispensing device,
blocking means configured to block the locking means in the unlocked position,
first disengagement means for disengaging the blocking means configured to be triggered upon the activation of the product dispensing device, and
second disengagement means for disengaging the blocking means configured to be triggered independently of the activation of the product dispensing device.

The system can therefore be relocked without having to deliver a dose of product which avoids wasting product for example because the system had been unlocked but finally delivery of a dose was not wanted. This is the case for example when accidental unlocking occurs. This is also advantageous since the locking means could be designed to return to the locked position according to a predetermined parameter independently of the activation of the dispensing device, for example according to a latency time that would be too long after unlocking or dispensing a dose of product or due to incorrect inclination of the dispensing device.

In one embodiment of the invention, the locking means can be integrated directly in the dispensing device, for example by interacting with a pump included in said dispensing device. In this case, the assistance device could be similar to a reusable device that could adapt to different successive dispensing devices comprising different types of locking means. In this case, the assistance device would be durable and highly practical.

Preferably, the actuator comprises at least one muscle wire.

The use of such a muscle wire offers several advantages. A muscle wire requires very low activation energy to switch the locking means from the locked position to the unlocked position. Furthermore, such a muscle wire is interesting since it is wear-resistant and will therefore not have to be replaced too often. In addition, its activation is highly reliable by controlling the electric current applied to it.

According to another embodiment, the actuator comprises a motor.

The use of a motor as actuator allows greater displacements of the locking means (for example by integrating a wheel or a rack controlled by this motor) compared with another means such as a muscle wire. Similarly, if a rotating motor is used, one direction of rotation of the motor could move the locking means towards the unlocked position while the other direction of rotation of the motor would return the locking means towards the locked position. Furthermore, control means capable of controlling and/or monitoring the actions of the motor could be provided.

Advantageously, the second disengagement means comprise at least one muscle wire.

As indicated previously, the use of such a muscle wire offers several advantages. A muscle wire requires very low activation energy to switch the locking means from the locked position to the unlocked position. Furthermore, such a muscle wire is interesting since it is wear-resistant and will therefore not have to be replaced too often. In addition, its activation is highly reliable by controlling the electric current applied to it.

Advantageously, the second disengagement means comprise a motor.

The use of a motor as second disengagement means allows greater displacements of the locking means (for example by integrating a wheel or a rack controlled by this motor) compared with another means such as a muscle wire. Furthermore, control means capable of controlling and/or monitoring the actions of the motor could be provided.

Preferably, the second disengagement means are configured to be activated after a predetermined time has elapsed from the switching of locking means to the unlocked position.

For example, this allows the locking means to switch automatically back to the locked position after unlocking has occurred. This is particularly advantageous when the locking means were switched to the unlocked position accidentally. This reduces the risk that a dispensing device which has been unlocked and left unattended could be used by a person for whom the product is not intended and for whom the product could represent a danger, for example a child. This also reduces the risk that product could be accidentally dispensed, for example in a bag or a pocket. Thus, this avoids wasting product and prevents the patient from being able to access the product if the device is designed to remain in the locked position for a certain period of time after dispensing a dose of product. For example, the assistance device could comprise control means capable of checking the conditions permitting activation of the second disengagement means. For example, these control means could comprise a detector to detect an unlocked position and/or a timer capable of measuring the time elapsed after unlocking the dispensing device.

Advantageously, the assistance device further comprises at least one inclination sensor for measuring the inclination of the dispensing device, the second disengagement means being configured to be activated when, with the locking means in the unlocked position, a measurement of the inclination of the dispensing device measured by the inclination sensor reaches a predetermined threshold during a predetermined period of time.

Thus, the locking means can be switched back to the locked position if the inclination measurement is equal to a predetermined threshold which indicates an inclination of the dispensing device that is incompatible with correct dispensing of the product. Generally, the inclination of the dispensing device can be measured by determining the angular offset between a main longitudinal axis of the dispensing device and an axis of gravity.

The inclination of the dispensing device could also be measured indirectly, for example by measuring an inclination of the assistance device and by deducing the inclination of the dispensing device since the angular offset between a main longitudinal axis of the assistance device and a main longitudinal axis of the dispensing device is constant and known for an assistance device associated with a given dispensing device. Preferably, the main longitudinal axes of the dispensing and assistance devices are parallel, or even coincide. The inclination threshold could be defined relative to any plane or axis, for example relative to a vertical axis or a horizontal axis.

According to an advantageous embodiment, the locking means are configured to switch from the unlocked position to the locked position by performing a rotational movement.

Thus, if for example the second disengagement means comprise a muscle wire, it is easier to use a longer muscle wire in order to obtain, for a given electric current, a longer stoke of the locking means during their rotation and therefore safer relocking while using low energy. The rotation of the locking means could be a rotation about a main longitudinal axis of the assistance device.

The locking means could also be configured to switch from the locked position to the unlocked position by performing a rotational movement. Alternatively, the locking means could be configured such that switching from the locked position to the unlocked position and/or switching from the unlocked position to the locked position occurs by a movement other than a rotational movement, for example by a translational movement or by a movement comprising at least one rotational component and at least one translational component.

Advantageously, the assistance device comprises at least one indentation capable of preventing the moving part of the dispensing device from returning to the rest position after a displacement along a predetermined stroke of the moving part of the dispensing device, the predetermined stroke being shorter than a total stroke corresponding to the activation position of the dispensing device.

Thus, this reduces the risk that the dispensing device should be partially activated and therefore that only part of the dose of product to be dispensed should actually be dispensed. The risk of such partial dispensing is that a user, for example a patient, should receive an insufficient dose of product. The indentation could have a shape complementary to that of an indentation present on the moving part of the dispensing device, these indentations being arranged to engage when the moving part of the dispensing device has travelled the predetermined stroke. The assistance device could comprise counting means for counting the number of doses of product that has been dispensed, these counting means being designed to consider that a dose of product has been dispensed only when the moving part of the dispensing device has travelled a longer stoke than the predetermined stroke and has preferably travelled the total stroke.

### BRIEF DESCRIPTION OF THE DRAWINGS

We will now describe several embodiments of the invention given as non-limiting examples referring to the attached figures, on which:

FIG. 1 is a perspective view of an assistance device and of a dispensing device according to a first embodiment of the invention.

FIG. 2A and FIG. 2B are respectively perspective and plan views of a part of the assistance device of the first embodiment, showing in particular locking means and blocking means of the locking means.

FIG. 3A through FIG. 3C are perspective views of a part of the assistance device of the first embodiment in which the locking means are respectively in the locked position, blocked in the unlocked position by the blocking means, and in the process of returning to the locked position.

FIG. 4A through FIG. 4H are cross-sectional views of an assistance device according to a second embodiment of the invention with the locking means in different locked and unlocked states.

FIG. 5A through FIG. 5H are cross-sectional views of an assistance device according to a third embodiment of the invention with the locking means in different locked and unlocked states.

FIG. 6 is an exploded perspective view of a part of an assistance device according to a fourth embodiment of the invention.

FIG. 7A through FIG. 7H are top plan views of an assistance device according to the fourth embodiment of the invention with the locking means in different locked and unlocked states.

FIG. 8 is an exploded perspective view of a part of an assistance device according to a fifth embodiment of the invention.

FIG. 9A through FIG. 9H are cross-sectional views of an assistance device according to the fifth embodiment of the invention with the locking means in different locked and unlocked states.

FIG. 10A through FIG. 9H are cross-sectional views of an assistance device according to a sixth embodiment of the invention with the locking means in different locked and unlocked states.

FIG. 11 is an exploded perspective view of a part of an assistance device according to a seventh embodiment of the invention.

FIG. 12A through FIG. 12H are cross-sectional views of an assistance device according to the seventh embodiment of the invention with the locking means in different locked and unlocked states.

### DETAILED DESCRIPTION OF THE INVENTION

The following embodiments describe assistance devices for the use of a product dispensing device, in which the dispensing device is a nasal spray and the product is a medication, in particular an opioid-based painkiller medication. The dispensing device could be any other dispensing device known by those skilled in the art, for example a device for dispensing liquid product in the form of drops. The product could also be any other product compatible for use with a device for dispensing a product, this product being a medication or not, for example a cosmetic product. In the following description, the same elements between the various embodiments are assigned a reference number in which only the hundreds digit changes.

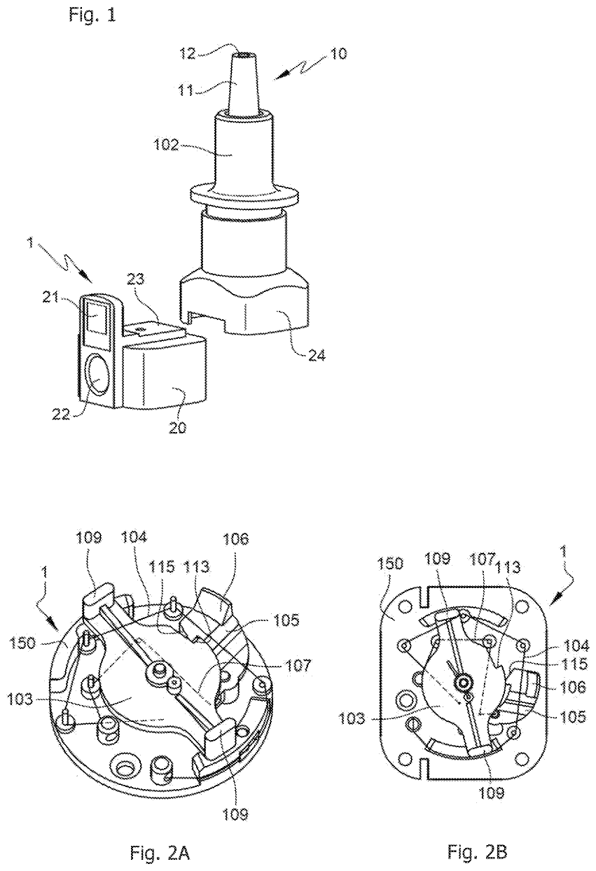

FIG. 1 through FIG. 3C show an assistance device 1 for the use of a product dispensing device 10 according to a first embodiment of the invention.

The dispensing device 10 to which the assistance device 1 is engaged comprises a pump 11 having a dispensing nozzle 12 and a reservoir (not shown). The dispensing device 10 further comprises a moving part 102 forming an applicator. This applicator 102 is mounted movably so as to be able to slide along a main longitudinal axis of the dispensing device 10 between a rest position, in which the applicator 102 is in its most distal position and therefore closest to the dispensing nozzle 12, and an activation position which is the minimum position that the applicator 102 must take such that the entire dose of product to be dispensed when the dispensing device 10 is activated is actually dispensed. It may be the most proximal position of the applicator 102, i.e. the farthest away from the dispensing nozzle 12. It could be another predetermined position, less proximal, i.e. nearer to the dispensing nozzle 12. Similarly, any other dispensing device 10 could be used in association with the assistance device 1 according to the invention, provided that its operation is compatible with that of the assistance device 1. The assistance device 1 would then represent a reusable device that could be assembled and interact successively with different dispensing devices 10, of the same or different type. These dispensing devices 10 would then form “disposable” devices that can be thrown away after each end of use. An end of use could consist of an end of treatment, an empty reservoir or a filling level that is insufficient to guarantee appropriate dispensing of the product.

In this embodiment, the assistance device 1 is made of several parts. The assistance device 1 could nevertheless be made of one part. A first part consists of a reusable socket 20. This socket 20 comprises in particular signalling means comprising a screen 21 capable of displaying a visual signal, a fingerprint reader 22 and electronic components (not shown) required to control and monitor the various functions of the assistance device 1 which will be detailed below. The socket 20 comprises an upper wall from which a pin 23 extends. The assistance device further comprises locking means 103 (see FIG. 2A and FIG. 2B) included in a base 24. The base 24 comprises a lower wall from which extends a complementary pin (not shown) of shape complementary to that of the pin 23 extending from the socket 20 and capable of engaging with it in order to engage the base 24 and the socket 20 and allow them to communicate electrically. The assistance device 1 also comprises engaging means (not shown) to the dispensing device 10.

The locking means 103 comprise an element having a general shape of a disc with two diametrically opposed portions that extend into a protuberance contained in the same plane as that containing the disc.

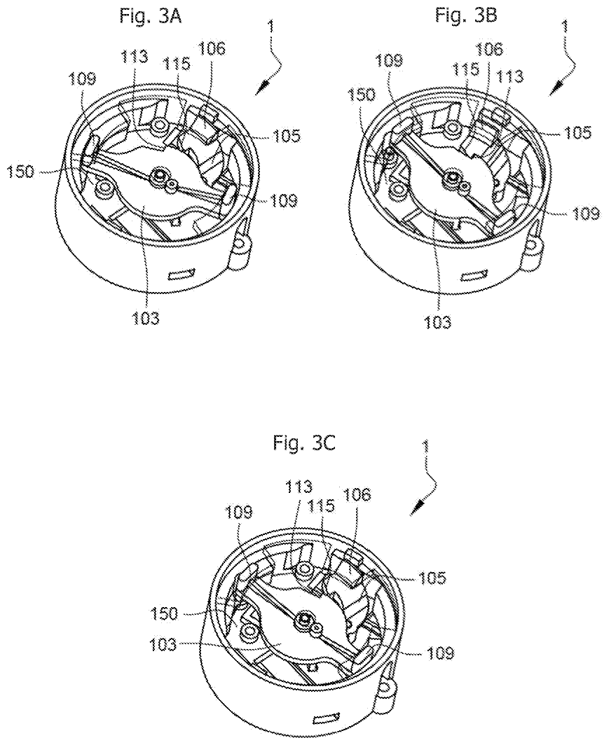

The locking means 103 comprise a stud 109 extending perpendicularly from each of the protuberances of the disc. There are therefore two of these studs 109. There could be a different number of protuberances extending from the disc, for example between one and ten, preferably between two and four. There could also be a different number of studs 109 extending from each protuberance, for example between one and ten, preferably between two and four. The studs 109 have a generally circular shape of oblong cross-section. They could have another shape, for example a shape of different cross-section such as square, rectangular, oval, round or parallelepipedic. The disc of the locking means 103 has a notch 113 formed around a periphery of the disc. The disc of the locking means 103 is attached to a plate 150 of the assistance device 1 and is mounted movable in rotation relative to it. Consequently, the studs 109 which form a single part with the disc are also mounted movable in rotation relative to the assistance device 1. This rotation takes place about an axis passing through the centre of the disc and perpendicular to the plane containing the disc. The locking means 103 are movable in rotation between a locked position in which the two studs 109 are each respectively against a stop of the assistance device 1 (see FIG. 3A) and an unlocked position in which the studs 109 of the locking means are not against the stop (see FIG. 2A and FIG. 3B). As described below, in the locked position, the locking means 103 prevent the dispensing of product by the dispensing device 10, and in the unlocked position, the locking means 103 do not prevent the dispensing of product.

The assistance device 1 further comprises an actuator 104 configured to switch the locking means 103 from the locked position to the unlocked position (see FIG. 2A and FIG. 2B). In the present case, the actuator 104 is a muscle wire. Muscle wires are well known by those skilled in the art and operate, when an electric current flows through them, by contracting by a length which depends on the strength of the electric current. One end of the muscle wire 104 is attached to the plate 150 and its other end is attached to the disc of the locking means 103. The muscle wire 104 contracts when current flows through it. Since the plate 150 is immobile, the end of the muscle wire 104 connected to the plate is also immobile while the end connected to the disc of the locking means 103, when the wire 104 contracts, rotates the locking means 103 such that they switch from the locked position to the unlocked position. The actuator 104 could be an element other than a muscle wire, for example a manual device, a solenoid or an electromagnet.

The assistance device 1 further comprises return means (not shown) configured to switch the locking means 103 from the unlocked position to the locked position. In the present case, the return means comprise a torsion spring which is stressed when the locking means 103 switch from the locked position to the unlocked position. Thus, these return means apply a force on the locking means 103 tending to return them to the locked position.

The assistance device 1 further comprises blocking means 105 configured to block the locking means 103 in the unlocked position. In the present case, the blocking means 105 have the general shape of a comma and are attached to the plate 150 so as to be movable in rotation by one of the ends of the comma. The blocking means 105 comprise an inner face directed towards the locking means 103 which is curved and whose curvature is complementary to that of the disc of the locking means 103, the curvature of the blocking means 105 facing that of the disc. This inner face of the blocking means 105 further comprises a complementary notch 115 capable of engaging with the notch 113 of the locking means 103 so as to hold the latter in the unlocked position despite the return force exerted by the return means.

The assistance device 1 further comprises first disengagement means 106 for disengaging the blocking means 105 configured to be triggered upon the activation of the dispensing device 10. The first disengagement means 106 consist of a protuberance extending from an upper face of the blocking means 105 and forming a single part with them. The first disengagement means 106 have an inner face opposite the locking means 103 and forming a ramp. These first disengagement means 106 are capable of disengaging the locking means 103 from the blocking means 105 such that the return means can exert their effect and therefore move the locking means 103 towards their locked position. The first disengagement means 106 are thus designed such that when an element exerting sufficient force moves along their ramp from top to bottom, i.e. from its free end towards its end attached to the blocking means 105, the first disengagement means 106 are moved and rotate the blocking means 105 with them such that the notch 113 and the complementary notch 115 of the locking means 103 and of the blocking means 105 are no longer engaged and therefore such that the locking means 103 return to the locked position under the effect of the return means. During the activation of the dispensing device 10, a part of the applicator 102 applies a force on the first disengagement means 106 and moves along their ramp from top to bottom in order to perform the above-mentioned disengagement.

The assistance device 1 also comprises second disengagement means 107 for disengaging the blocking means 105 configured to be triggered independently of the activation of the product dispensing device 10. In the present case, these second disengagement means 107 comprise a muscle wire. This muscle wire 107 is of the same type as the muscle wire 104 used as actuator to switch the locking means 103 from the locked position to the unlocked position. One end of the muscle wire 107 is attached to the plate 150 and its other end is attached to the blocking means 105. This muscle wire 107 contracts when an electric current flows through it. Since its end attached to the plate 150 is immobile, it pulls on its other end which is attached to the blocking means 105 and rotates them such that, when the locking means 103 and the blocking means 105 are engaged via the notch 113 and the complementary notch 115, the blocking means 105 move away from the locking means 103, in particular at the notch 113 and the complementary notch 115 such that they are no longer engaged. The locking means 103 are then rotated by the return means which move them to the locked position. Activation of the muscle wire 107 depends only on the current flowing through it and is therefore independent of an activation of the dispensing device 10. These second disengagement means 107 could be different from a muscle wire and could consist of an element capable of moving the blocking means 105, for example a motor and/or one or more magnets and/or one or more electromagnets.

The assistance device 1 also comprises a pin (not shown) having at least one complementary indentation of shape complementary to at least one indentation present on a part of the applicator 102. These at least one indentation and one complementary indentation are designed to prevent partial activation of the dispensing device 10. Thus, from the time when the applicator 102 has travelled a first predetermined stroke sufficient to start dispensing product, and until the applicator 102 has travelled a second predetermined stroke, corresponding to at least a stroke necessary to deliver a complete dose of product, the moving part of the dispensing device 10 via the applicator 102 cannot return to its rest position until the applicator 102 has travelled at least the second predetermined stroke.

In a preferred configuration, the assistance device 1 comprises an assembly part (not shown) forming a cover covering the locking means 103 and reinforcing the assembly formed by the locking and unlocking system in addition to stabilising it. This assembly part would be assembled with the locking means in order to be engaged thereto and to restrict their access to all users.

We will now describe a mode of operating the assistance device 1 according to this first embodiment.

In the rest state, the dispensing device 10 is in its rest position and the locking means 103 are in the locked position (see FIG. 3A). In the locked position, the studs 109 of the locking means 103 are opposite protuberances present on the applicator 102. These protuberances of the applicator 102 come up against the studs 109 when the user tries to use the dispensing device 10 without having unlocked it, which prevents the activation of the dispensing device 10. When the user wants to use the device, he places his finger on the fingerprint reader 22 to unlock it, it being understood that his fingerprint has previously been saved such that he can be recognised as an authorised user by the assistance device 1. When the user's fingerprint has been authenticated, electronic control means via the electronic components present in the socket 20 control the production of an electric current and cause it to flow through the muscle wire 104 forming the actuator such that it contracts. As it contracts, the muscle wire 104 switches the locking means 103 from the locked position (FIG. 3A) to the unlocked position (FIG. 3B) by rotating these locking means 103 clockwise relative to FIG. 2A through FIG. 3C. As the locking means 103 rotate, the complementary notch 115 of the blocking means 105 moves around the outside of the disc of the locking means 103 until it engages with the notch 113 of the locking means 103 (see FIG. 3B). Thus, the locking means 103 are blocked in the locked position by the blocking means 105. In the unlocked position, the protuberances of the applicator 102 are no longer opposite the studs 109 of the locking means 103 which have been moved during the rotation of the locking means 103. Thus, the protuberances are no longer against the studs 109 when a user tries to activate the dispensing device 10. The dispensing device 10 can therefore be activated.

Two options are then possible: the user either activates the dispensing device 10, or does not.

If the user activates the dispensing device 10, the user moves the applicator 102 in the proximal direction, i.e. downwards in reference to FIG. 1. During the activation, the applicator 102 is moved towards the activation position of the dispensing device 10. During this displacement, a portion of the applicator 102 (not shown) applies a pressure on the first disengagement means 106 by moving along their ramp. The assistance device 1 is designed such that when the portion of the applicator 102 moves along the ramp of the first disengagement means 106, the blocking means 105 and the locking means 103 can only be disengaged when the activation position has been reached, to prevent the dispensing device 10 from relocking although a complete dose has not been delivered. Thus, when the portion of the applicator 102 has travelled the predetermined stroke, the locking means 103 and the blocking means 105 are disengaged from each other and the return means return the locking means 103 to the locked position (see FIG. 3C).

Detection means can be included in the assistance device 1 to detect the number of doses dispensed by the device. These detection means may comprise at least one contact rod intended to be against the blocking means 105 when the latter have been disengaged from the locking means 103 by the first disengagement means 106, when the activation position has been reached. The contact between the contact rod and the blocking means 105 provides the following information: the activation position is reached and therefore a dose of product has been dispensed and the blocking means 105 have been disengaged. This information confirming that a dose of product has been correctly dispensed can be transmitted to the electronic control means to monitor and control the dispensing device and the dispensing of the product.

If the user does not activate the dispensing device 10, either because unlocking occurred accidentally, or because the user finally does not want to obtain a dose of product, it must be possible to relock the device, in particular to prevent an unauthorised person from using it, without however activating the dispensing device 10, in particular to avoid wasting product. To do this, a button for example and electronic control means present in the socket 20 are used to trigger the flow of an electric current in the second disengagement means 107 formed by the muscle wire. As it contracts, this muscle wire 107 rotates the blocking means 105 such that their notch 115 is no longer engaged with the notch 113 of the locking means 103. The locking means 103 thus released are returned to the locked position by the return means without having to activate the dispensing device 10. As an alternative or in addition, the disengagement of the locking means 103 could be controlled differently. For example, the assistance device 1 could comprise a chronometer which is started when the locking means 103 switch from the locked position to the unlocked position and the electronic control means via the electronic components present in the socket 20 could be configured to trigger the flow of an electric current in the muscle wire 107 when the chronometer reaches a predetermined value. As an alternative or in addition, the assistance device 1 and/or the dispensing device 10 could also comprise a sensor to measure the inclination of the assistance device 1 and/or of the dispensing device 10 and the electronic control means could be configured to trigger the flow of an electric current in the muscle wire 107 when the inclination of the assistance device 1 and/or of the dispensing device 10 measured by the inclination sensor reaches or exceeds a predetermined threshold.

In the embodiments described below, the same elements between the embodiments have the same name as in the first embodiment and perform the same function despite their shape or type which may be completely different.

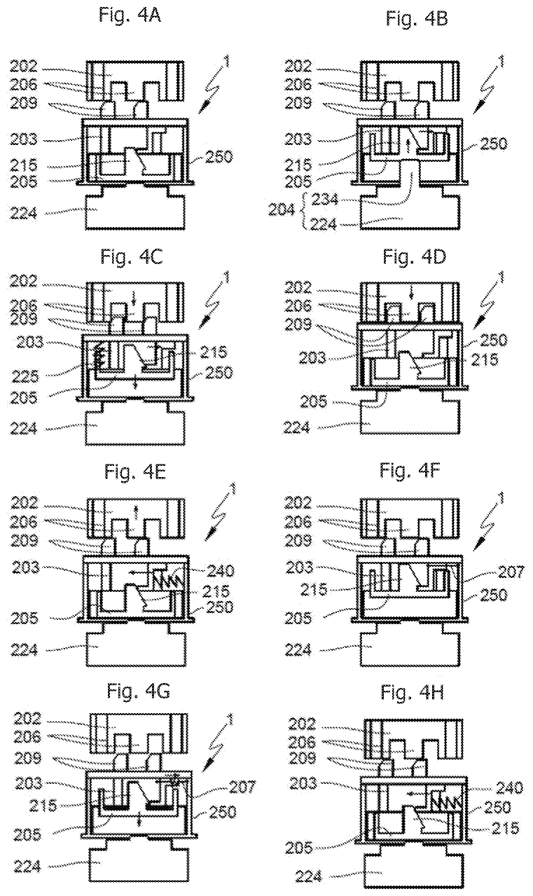

FIG. 4A through FIG. 4H show an assistance device 1 for assisting in the use of a product dispensing device 10 according to a second embodiment of the invention.

In this embodiment, the locking means 203 have the general shape of a parallelepiped and comprise studs 209 which extend from two opposite ends. The studs 209 also have the general shape of a parallelepiped but each one has a chamfer forming a ramp. The ramps of all the studs 209 are oriented in the same direction and have the same angle of inclination (see FIG. 4A through FIG. 4H). In the present case, there are four studs 209, two at each of the two opposite ends of the locking means 203. There could be a different number of studs 209, for example between two and twenty, preferably between four and eight, or even between four and six. The locking means 203 are mounted on the assistance device 1 such that they can slide horizontally, i.e. in the direction of an axis perpendicular to a main longitudinal axis of the assistance device 1. The locking means 203 have openings in a lower wall of the parallelepiped they form.

The blocking means 205 also have the general shape of a parallelepiped with overall dimensions greater than those of the locking means 203 such that the blocking means 205 are capable of containing at least partly the locking means 203. The blocking means 205 comprise two protuberances forming clips 215 extending from a main upper surface of the blocking means 205. The clips 215 are movable in vertical translation, i.e. along a main longitudinal axis of the assistance device 1 but are immobile in horizontal translation, i.e. along an axis perpendicular to the main longitudinal axis of the assistance device 1. In the rest position, the clips 215 are not engaged with the locking means 203 and ends of a lower wall of the locking means 203 are against an inclined ramp of each clip 215 (see FIG. 4A). In the blocked position, the clips 215 are engaged with the locking means 203 and prevent them from returning to the locked position. A spring 225 is compressed when the blocking means 205 switch from the rest position to the blocked position and therefore applies a force tending to return the blocking means 205 to the rest position. For clarity purposes, this spring 225 is only shown on FIG. 4C.

In this embodiment, the actuator 204 comprises a base 224 and a pin 234 movable in vertical translation relative to this base 224 between a retracted position in which the pin 234 is completely or almost completely inside the base 224 and an extended position in which the pin 234 forms a projection from an upper wall of the base 224. In the extended position, the pin 234 has a main longitudinal axis which is perpendicular to the plane containing the upper wall of the base 234 from which the pin 234 extends.

A portion of the proximal end of the applicator 202 forms crenelations comprising peaks and troughs. The troughs are configured to receive the studs 209 of the locking means 203. Some of the peaks of the applicator 202 form the first disengagement means 206. Thus, the locking means 203 are mounted such that they can slide horizontally between a locked position (see FIG. 4A) in which the tops of the studs 209 are opposite one of the peaks of the proximal end of the applicator 202 and an unlocked position (see FIG. 4B) in which the tops of the studs 209 are opposite the troughs of the proximal end of the applicator 202 while the ramps of the studs 209 are opposite the peaks of the proximal end of the applicator 202.

The return means 240 configured to switch the locking means 203 from the unlocked position to the locked position are shown in this case by a compressed spring (shown only on FIG. 4E and FIG. 4H for clarity purposes). This spring 240 is compressed when the locking means 203 switch from the locked position to the unlocked position and therefore applies a force tending to return them to the locked position.

The second disengagement means 207 comprise in this case a muscle wire having one end attached to a casing 250 containing the locking means 203 and the blocking means 205. The other end of the muscle wire 207 is attached to the locking means 203. Thus, by contracting under the effect of an electric current, the muscle wire 207 is capable of moving the locking means 203 in horizontal translation towards the unlocked position. For clarity purposes, the second disengagement means 207 are only shown on FIG. 4F and FIG. 4G.

We will now describe a mode of operating the assistance device 1 according to this second embodiment.

In the rest state, the moving part of the dispensing device 10 via the applicator 202 is in its rest position, the locking means 203 are in the locked position and the blocking means 205 are in the rest position (see FIG. 4A). As indicated previously, in the locked position, the studs 209 of the locking means 203 are opposite peaks of the proximal end of the applicator 202. These peaks of the applicator 202 come up against the tops of the studs 209 when the user tries to use the dispensing device 10 without having unlocked it, which prevents the activation of the dispensing device 10. When the user wants to use the dispensing device 10, he places his finger on the fingerprint reader 22 to unlock it, it being understood that his fingerprint has previously been saved such that he can be recognised as an authorised user by the assistance device 1. When the user's fingerprint has been authenticated, the electronic control means via the electronic components present in the socket 20 instruct the actuator 204 to make the pin 234 come out of the base 224. As it comes out, the pin 234 moves the blocking means 205 in vertical translation, i.e. along the main longitudinal axis of the assistance device 1, in the distal direction, i.e. towards the dispensing device 10. During this vertical translation of the blocking means 205, the lower wall of the locking means 203 which is against the ramp of the clips 215 moves along this ramp. Since the immobilising means 205 are not movable in horizontal translation, as the lower wall of the locking means moves along the ramp of the clips 215, the locking means 203 are moved in horizontal translation such that they switch to the unlocked position and, at the end of the ramp, the clips 215 engage with the lower wall of the locking means 203 (see FIG. 4B). Thus, the locking means 203 are blocked in the unlocked position by the blocking means 205. In the unlocked position, the peaks of the end of the applicator 202 are no longer opposite the tops of the studs 209 of the locking means 203 but opposite the ramps of these studs 209. Thus, these protuberances are no longer against the tops of the studs 209 when a user tries to activate the dispensing device 10, but move along the ramp of each stud 209 (see FIG. 4C). The dispensing device 10 can therefore be activated.

Two options are then possible: the user either activates the dispensing device 10, or does not.

If the user activates the dispensing device 10, the user moves the actuator 202 in the proximal direction, i.e. downwards in reference to FIG. 4A through FIG. 4H. During the activation, the applicator 202 is moved towards the activation position of the dispensing device 10. During this displacement, the peaks of the end of the applicator 202 move along the inclined ramps of the studs 209 (see FIG. 4C). Since the applicator 202 is not movable in horizontal translation, the locking means 203 are moved in horizontal translation such that they are no longer engaged with the clips 215 and such that, as the applicator 202 moves downwards, the studs 209 engage in its troughs. Since the blocking means 205 are no longer engaged with the locking means 203, they are returned to the rest position by the spring 225 (see FIG. 4A and FIG. 4D). When the activation is finished, the applicator 202 moves back up to the rest position and the return means 240 switch the locking means 203 to the locked position (see FIG. 4E). We are therefore back to the initial situation, i.e. the locking means 203 are in the locked position and the dispensing device 10 and the blocking means 205 are in the rest position.

If the user does not activate the dispensing device 10, either because unlocking occurred accidentally, or because the user finally does not want to obtain a dose of product, it must be possible to relock the dispensing device 10, in particular to prevent an unauthorised person from using it, without however activating the dispensing device 10, in particular to avoid wasting product. To do this, a button for example and electronic control means present in the socket 20 are used to trigger the flow of an electric current in the second disengagement means 207 formed by the muscle wire (see FIG. 4F). As it contracts, this muscle wire 207 moves the locking means in horizontal translation such that they are no longer engaged with the blocking means 205 (see FIG. 4G). Thus, the spring 225 moves the blocking means 205 in vertical translation downwards to return them to the rest position and the return means 240 formed by the spring move the locking means 203 in horizontal translation up to the locked position (see FIG. 4H) without having to activate the dispensing device 10. As indicated previously, as an alternative or in addition, the disengagement of the locking means 203 could be controlled differently.

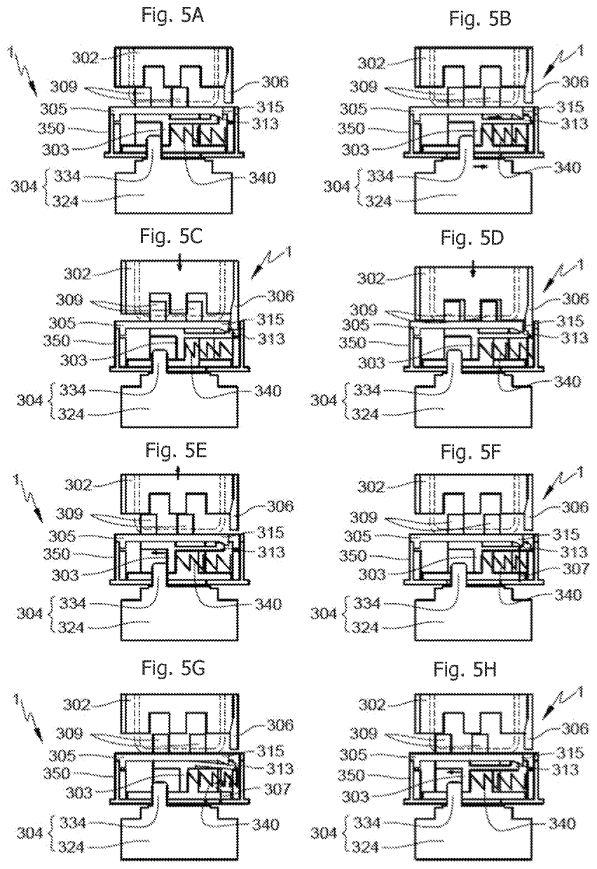

FIG. 5A through FIG. 5H show an assistance device 1 for the use of a product dispensing device 10 according to a third embodiment of the invention.

In this embodiment, the locking means 303 have the general shape of a parallelepiped and comprise studs 309 which extend from two opposite ends. The studs 309 also have the general shape of a parallelepiped. In the present case, there are four studs 309, two at each of the two opposite ends of the locking means 303. There could be a different number of studs 309, for example between two and twenty, preferably between four and eight, or even between four and six. The locking means 303 are mounted on the assistance device 1 such that they can slide horizontally, i.e. in the direction of an axis perpendicular to the main longitudinal axis of the assistance device 1. The locking means 303 have openings in a lower wall of the parallelepiped they form. The locking means 303 comprise a lateral projection. A lug 313 extends from one free end of this projection.

The blocking means 305 have the general shape of a disc. The blocking means 305 could have any other shape compatible with a use of the assistance device 1. A complementary lug 315 extends from a part of a peripheral portion of the blocking means 305 towards the lateral projection of the locking means 303. The blocking means 305 are mounted so as to be immobile with respect to the assistance device 1. In the rest position, the lug 313 of the locking means 303 and the complementary lug 315 of the blocking means 305 are not engaged and are away from each other (see FIG. 5A). In the blocked position, the lug 313 of the locking means 303 is against the complementary lug 315 of the blocking means 305 such that the blocking means 305 prevent the locking means 303 from moving in horizontal translation towards the locked position (see FIG. 5B).

The return means 340 configured to switch the locking means 303 from the unlocked position to the locked position are shown in this case by a compressed spring. This spring 340 is compressed when the locking means 303 switch from the locked position to the unlocked position and therefore applies a force tending to return them to the locked position.

In this embodiment, the actuator 304 comprises a base 324 and a pin 334 movable in horizontal translation relative to this base 324 between a rest position (see FIG. 5A) and an activation position (see FIG. 5B). The pin 334 forms a projection from an upper wall of the base 324 and has a main longitudinal axis which is perpendicular to the plane containing the upper wall of the base 324 from which the pin 334 extends. In the rest position, the pin 334 extends inside the locking means 303 through an opening in their lower wall and rests against an inner wall of the locking means 303 (see FIG. 5A). The pin 334 is capable of moving the locking means 303 in translation by pressing against this inner wall and thus switches them from the locked position to the unlocked position (see FIG. 5B).

The applicator 302 comprises a protuberance forming the first disengagement means 306. This protuberance is formed by an extension of the wall of the applicator at its proximal end. When the locking means 303 are blocked in the unlocked position as shown on FIG. 5B, this protuberance 306 is capable of applying a force on the lug 313 of the locking means 303 so as to disengage it from the complementary lug 315 of the blocking means 305 (see FIG. 5C and FIG. 5D).

A portion of the proximal end of the applicator 302 forms crenelations comprising peaks and troughs. The troughs are configured to receive the studs 309 of the locking means 303. Thus, the locking means 303 are mounted such that they can slide horizontally between a locked position (see FIG. 5A) in which the tops of the studs 309 are each opposite one of the peaks of the proximal end of the applicator 302 and an unlocked position (see FIG. 5B) in which the tops of the studs 309 are each opposite one of the troughs of the proximal end of the applicator 302.

The second disengagement means 307 comprise in this case a muscle wire having one end attached to a casing 350 containing the locking means 303 and the blocking means 305. The other end of the muscle wire 307 is attached to the lateral projection of the locking means 303. Thus, by contracting under the effect of an electric current, the muscle wire 307 is capable of deforming this lateral projection such that the lug 313 of the locking means 303 is no longer engaged with the complementary lug 315 of the blocking means 305. For clarity purposes, the second disengagement means 307 are only shown on FIG. 5F and FIG. 5G.

We will now describe a mode of operating the assistance device 1 according to this third embodiment.

In the rest state, the dispensing device 10 is in its rest position, the locking means 303 are in the locked position and the blocking means 305 are in the rest position (see FIG. 5A). As indicated previously, in the locked position, the studs 309 of the locking means 303 are opposite peaks of the proximal end of the applicator 302. These peaks of the applicator 302 come up against the tops of the studs 309 when the user tries to use the dispensing device 10 without having unlocked it, which therefore prevents the activation of the dispensing device 10. When the user wants to use the dispensing device 10, he places his finger on the fingerprint reader 22 to unlock it in the same way as for the previous embodiments. When the user's fingerprint has been authenticated, the electronic control means via the electronic components present in the socket 20 instruct the actuator 304 to move the pin 334 in horizontal translation from its rest position to its activation position. During this translation, the pin 334, which is against the inner wall of the locking means 303, moves them in horizontal translation from their locked position to their unlocked position (see FIG. 5A and FIG. 5B). The lug 313 of the locking means 303 has a ramp facing towards the lug 315 of the blocking means 305. The complementary lug 315 of the blocking means 305 has a complementary ramp facing towards the lug 313 of the locking means 303. During the translation of the locking means 303, the ramp of the lug 313 of the locking means 303 moves along the ramp of the complementary lug 315 of the blocking means 305, the latter ramp being fixed relative to the assistance device 1, such that the lateral projection of the locking means 303 progressively deforms elastically downwards as the locking means 303 translate towards the unlocked position under the effect of the pin 334. When the end of the ramp of the complementary lug 315 has been reached, the lateral projection returns to its initial conformation such that the lug 313 and the complementary lug 315 are against each other such that the blocking means 305 prevent the locking means 303 from moving towards the locked position (see FIG. 5B). The locking means 303 are thus blocked in the unlocked position.

In the unlocked position, the peaks of the end of the applicator 302 are no longer opposite the tops of the studs 309 of the locking means 303. Thus, the peaks of the end of the applicator 302 no longer come up against the tops of the studs 309 when a user tries to activate the dispensing device 10. The dispensing device 10 can therefore be activated (see FIG. 5C).

Two options are then possible: the user either activates the dispensing device 10, or does not.

If the user activates the dispensing device 10, the user moves the actuator 302 in the proximal direction, i.e. downwards in reference to FIG. 5A through FIG. 5H. During the activation, the applicator 302 is moved towards the activation position of the dispensing device 10. As the applicator 302 moves downwards, the studs 309 engage in its troughs. After a predetermined stroke, the first disengagement means 306 reach the lug 313 of the locking means 303 and apply a force on them so as to elastically deform the lateral projection of the locking means 303 (see FIG. 5D) and thus release the lug 313 of the locking means 303 from the complementary lug 315 of the blocking means 305. When the lug 313 and the complementary lug 315 are no longer against each other, the spring of the return means 340 pushes the locking means 303 such that they slide horizontally up to their locked position (see FIG. 5E). We are therefore back to the initial situation, i.e. the locking means 303 are in the locked position and the dispensing device 10 and the blocking means 305 are in the rest position.

If the user does not activate the dispensing device 10, either because unlocking occurred accidentally, or because the user finally does not want to obtain a dose of product, it must be possible to relock the dispensing device 10, in particular to prevent an unauthorised person from using it, without however activating the dispensing device 10, in particular to avoid wasting product. To do this, a button for example and electronic control means present in the socket 20 are used to trigger the flow of an electric current in the second disengagement means 307 formed by the muscle wire (see FIG. 5F). By contracting, this muscle wire 307 elastically deforms the lateral projection of the locking means 303 downwards so as to disengage the lug 313 from the complementary lug 315 (see FIG. 5G). Thus, the return means 340 move the locking means 303 in horizontal translation up to the locked position (see FIG. 5H) without having to activate the dispensing device 10. As indicated previously, as an alternative or in addition, the disengagement of the locking means 303 could be controlled differently.

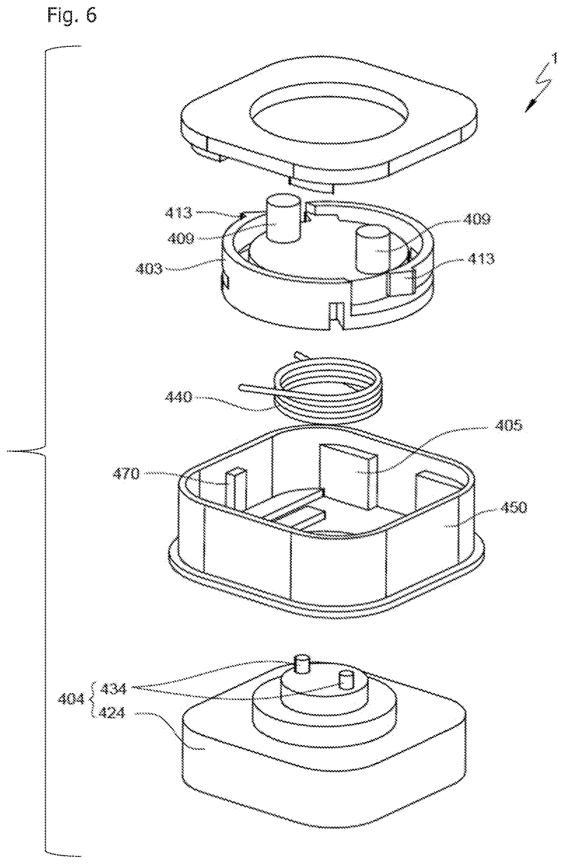

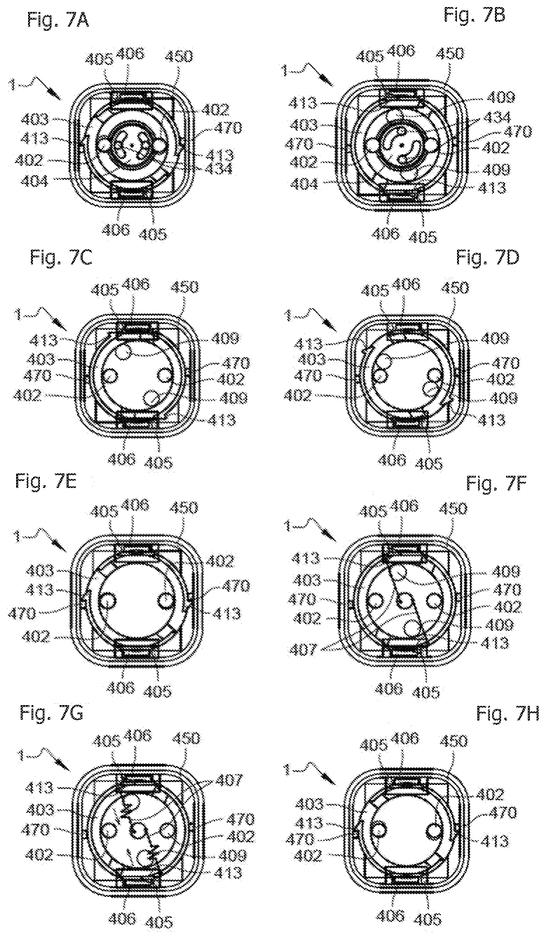

FIG. 6 through FIG. 7H show an assistance device 1 for use of a product dispensing device 10 according to a fourth embodiment of the invention.

In this embodiment, the locking means 403 have the general shape of a circular cylinder and comprise studs 409 which extend from an upper wall of the locking means 403. The studs 409 also have the general shape of a circular cylinder (see FIG. 6). In the present case, there are two diametrically opposed studs 409. There could be a different number of studs 409, for example between two and twenty, preferably between two and eight, or even between two and six. The locking means 403 are mounted on the assistance device 1 such that they can rotate about a main longitudinal axis of the assistance device 1. The locking means 403 comprise two circumferential lateral projections each extending on a part of the periphery of the locking means 403. A lug 413 extends from one free end of each of these lateral projections.

The assistance device 1 comprises a casing 450 capable of containing the locking means 403. The blocking means 405 consist of reliefs extending from an inner face of a lateral wall of the casing 450. In this configuration, the blocking means 405 are therefore mounted so as to be immobile with respect to the assistance device 1. The casing 450 further comprises two reliefs 470 extending respectively on two inner faces of the lateral walls of the casing 450, these walls being different from those from which the blocking means 405 extend. In the rest position, the lug 413 of the locking means 403 and the reliefs of the blocking means 405 are not engaged and are away from each other (see FIG. 7A). Each lug 413 of the locking means 403 then comes up against one of the reliefs 470 (see FIG. 7A). In the blocked position, the lugs 413 of the locking means 403 are each against a flat face of one of the reliefs of the blocking means 405 such that the blocking means 405 prevent the locking means 403 from rotating towards the locked position (see FIG. 7B).

The return means 440 configured to switch the locking means 403 from the unlocked position to the locked position are shown in this case by a spring under tension. This spring 440 is compressed when the locking means 403 switch from the locked position to the unlocked position and therefore applies a force tending to return them to the locked position.

In this embodiment, the actuator 404 comprises a base 424 and two pins 434 movable in rotation relative to this base 424 about the main longitudinal axis of the assistance device 1 between a rest position (see FIG. 7A) and an activation position (see FIG. 7B). The pins 434 form a projection from an upper wall of the base 424 and have each a main longitudinal axis which is perpendicular to the plane containing the upper wall of the base 434 from which they extend. The pins 434 extend inside the locking means 403 through an opening in their lower wall. The pins 434 are capable of rotating the locking means 403 about the main longitudinal axis of the applicator 402 and thus switch them from the locked position to the unlocked position.

The applicator 402 comprises two protuberances forming the first disengagement means 406. These protuberances are formed by an extension of the wall of the applicator 402 at its proximal end. When the locking means 403 are blocked in the unlocked position as shown on FIG. 7B, these protuberances 406 are capable of applying a force on the lug 413 of the locking means 403 so as to disengage the lugs 413 from the reliefs of the blocking means 405 by elastically deforming the lateral projections of the locking means 403 (see FIG. 7C).

The applicator 402 comprises studs that have the general shape of a circular cylinder and substantially the same diameter as the studs 409 of the locking means 403. The locking means 403 are mounted movable in rotation between a locked position (see FIG. 7A) in which the tops of the studs 409 are each opposite the top of one of the respective studs of the applicator 402 and an unlocked position (see FIG. 7B) in which the tops of the studs 409 of the locking means 403 are not opposite the top of the studs of the applicator 402.

In the present case, the second disengagement means 407 comprise two muscle wires, one end of each of these muscle wires 407 being attached to a central portion of the locking means 403. The other end of each of these muscle wires 407 is attached to one of the respective lateral projections of the locking means 403. Thus, by contracting under the effect of an electric current, the muscle wires 407 are capable of elastically deforming these lateral projections such that the lugs 413 of the locking means 403 are no longer engaged with the reliefs of the blocking means 405. For clarity purposes, the second disengagement means 407 are only shown on FIG. 7F and FIG. 7G.

We will now describe a mode of operating the assistance device 1 according to this fourth embodiment.

In the rest state, the dispensing device 10 is in its rest position and the locking means 403 are in the locked position (see FIG. 7A). As indicated previously, in the locked position, the studs 409 of the locking means 403 are opposite studs of the applicator 402. These studs of the applicator 402 come up against the tops of the studs 409 when the user tries to use the dispensing device 10 without having unlocked it, which prevents the activation of the dispensing device 10. When the user wants to use the dispensing device 10, he places his finger on the fingerprint reader 22 to unlock it in the same way as for the previous embodiments. When the user's fingerprint has been authenticated, the electronic control means via the electronic components present in the socket 20 instruct the actuator 404 to rotate the pins 434 from their rest position to their activation position. During this rotation, the pins 434 move the locking means 403 from their locked position to their unlocked position (see FIG. 7A and FIG. 7B). The lugs 413 of the locking means 403 each have a ramp facing towards one of the respective reliefs of the blocking means 405. During the rotation of the locking means 403, one of the reliefs of the blocking means 405 moves along each respective ramp of the lugs 413 of the locking means 403. Since the reliefs of the blocking means 405 are fixed relative to the assistance device 1, the lateral projections of the locking means 403 progressively deform elastically towards the centre of the locking means 403 as the locking means 403 turn under the effect of the rotation of the pins 434. When the end of each ramp of the lugs 413 has been reached, each lateral projection of the locking means 403 returns to its initial conformation such that the lugs 413 of the locking means 403 and the reliefs of the blocking means 405 are against each other such that the blocking means 405 prevent the locking means 403 from moving towards the locked position under the action of the torsion spring 440 (see FIG. 7B). The locking means 403 are thus blocked in the unlocked position.

In the unlocked position, the studs of the applicator 402 are no longer opposite the tops of the studs 409 of the locking means 403. Thus, these studs are no longer against these tops when a user tries to activate the dispensing device 10. The dispensing device 10 can therefore be activated.

Two options are then possible: the user either activates the dispensing device 10, or does not.

If the user activates the dispensing device 10, the user moves the actuator 402 in the proximal direction, i.e. downwards in reference to FIG. 6. During the activation, the applicator 402 is moved towards the activation position of the dispensing device 10. After a predetermined stroke, the first disengagement means 406 reach the studs 413 of the locking means 403 and apply a force so as to elastically deform the lateral projections of the locking means 403 (see FIG. 7C). When each lug 413 of the locking means 403 and each relief of the blocking means 405 are no longer against each other, the torsion spring of the return means 440 rotates the locking means 403 up to their locked position (see FIG. 7E). We are therefore back to the initial situation, i.e. the locking means 403 are in the locked position and the dispensing device 10 is in the rest position.

If the user does not activate the dispensing device 10, either because unlocking occurred accidentally, or because the user finally does not want to obtain a dose of product, it must be possible to relock the dispensing device 10, in particular to prevent an unauthorised person from using it, without however activating the dispensing device 10, in particular to avoid wasting product. To do this, a button for example and electronic control means present in the socket 20 are used to trigger the flow of an electric current in the second disengagement means 407 formed by the muscle wires (see FIG. 7F). By contracting, these muscle wires 407 elastically deform the lateral projections of the locking means 403 towards the centre of the locking means 403 so as to disengage the lugs 413 of the locking means 403 from the reliefs of the blocking means 405 (see FIG. 7G). Thus, the return means 440 rotate the locking means 403 to the locked position (see FIG. 7H) without having to activate the dispensing device 10. As indicated previously, as an alternative or in addition, the disengagement of the locking means 403 could be controlled differently.

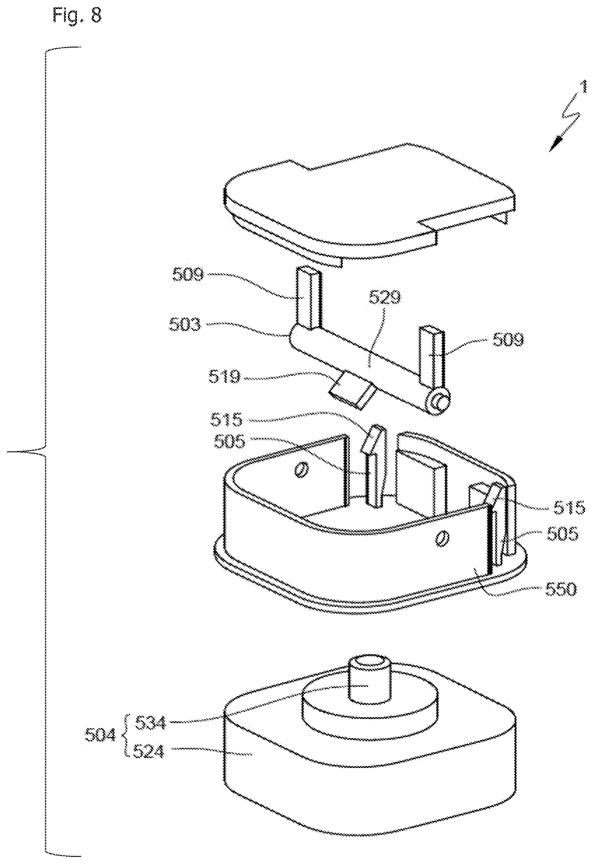

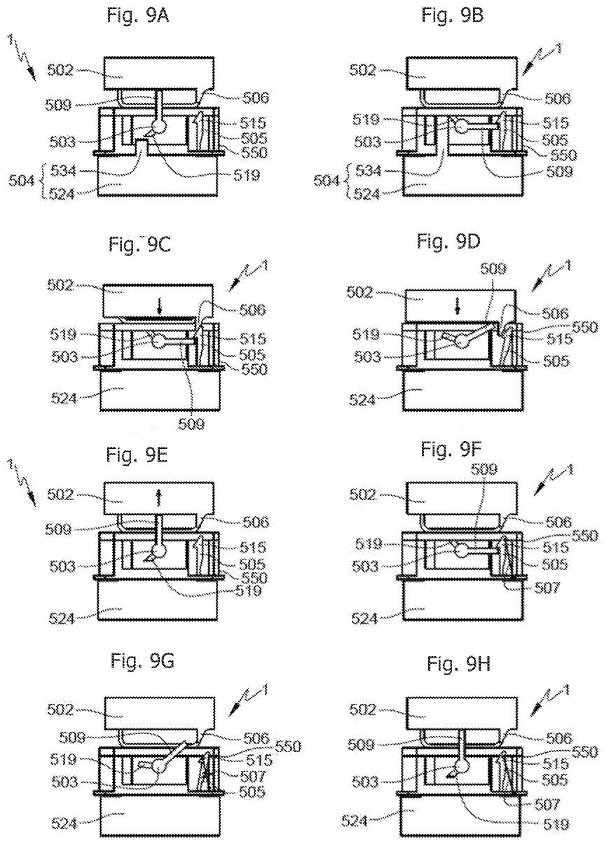

FIG. 8 through FIG. 9H show an assistance device 1 for in the use of a product dispensing device 10 according to a fifth embodiment of the invention.

In this embodiment, the locking means 503 have the general shape of a circular cylinder from which studs 509 extend from each end of a cylinder 529 perpendicular to a main longitudinal axis of the cylinder 529 (see FIG. 8). The studs 509 have the general shape of a parallelepiped. In the present case, there are two studs 509. There could be a different number of studs 509, for example between two and twenty, preferably between two and eight, or even between two and six. The locking means 503 further comprise a protuberance 519 extending radially from a median portion of the cylinder 529. The locking means 503 are mounted on the assistance device 1 such that they can rotate about an axis perpendicular to the main longitudinal axis of the assistance device 1.

The assistance device 1 comprises a casing 550 capable of containing the locking means 503. The blocking means 505 comprise two protuberances extending from an inner face of a lower wall of the casing 550 and each having a lug 515 on their free end. In the rest position, the studs 509 of the locking means 503 and the studs 515 of the blocking means 505 are not engaged and are away from each other (see FIG. 9A). In the blocked position, the studs 509 of the locking means 503 are against one of the respective lugs 515 of the blocking means 505 such that the blocking means 505 prevent the locking means 503 from rotating towards the locked position (see FIG. 9B).

The return means (not shown) configured to switch the locking means 503 from the unlocked position to the locked position comprise in this case a torsion spring. This spring is compressed when the locking means 503 switch from the locked position to the unlocked position and therefore applies a force tending to return them to the locked position.

In this embodiment, the actuator 504 comprises a base 524 and a pin 534 movable in vertical translation relative to this base 524 between a rest position (see FIG. 9A) and an activation position (see FIG. 9B). The pin 534 forms a projection from an upper wall of the base 524 and has a main longitudinal axis which is perpendicular to the plane containing the upper wall of the base 524 from which it extends. The pin 534 is capable of rotating the locking means 503 about their main longitudinal axis and thus switch them from the locked position to the unlocked position. To do this, the pin 534 pushes on the protuberance 519 of the locking means 503 (see FIG. 9B). The actuator 504 could be any other element capable of performing this function. For example, the actuator 504 could be a muscle wire capable, when it contracts, of rotating the locking means 503 from the locked position to the unlocked position.

The applicator 502 comprises two protuberances forming the first disengagement means 506. These protuberances 506 are formed by an extension of the wall of the applicator at its proximal end. When the locking means 503 are blocked in the unlocked position by the blocking means 505 as shown on FIG. 9B, these protuberances 506 are capable of applying a force on each of the lugs 515 of the blocking means 505 so as to disengage them from the studs 509 of the locking means 503 by elastically deforming the blocking means 505 (see FIG. 9C and FIG. 9D).

The locking means 503 are mounted movable in rotation between a locked position (see FIG. 9A) in which the tops of the studs 509 are each opposite a top of a proximal end of the applicator 502 and an unlocked position (see FIG. 9B) in which the tops of the studs 509 are not opposite a top of the proximal end of the applicator 502.

In the present case, the second disengagement means 507 comprise two muscle wires, one end of each of these muscle wires 507 being attached to the casing 550. The other end of each of these muscle wires 507 is attached to one of the respective lugs 515 of the blocking means 505. Thus, by contracting under the effect of an electric current, the muscle wires 507 are capable of elastically deforming these blocking means 505 such that the studs 509 of the locking means 503 are no longer engaged with the lugs 515 of the blocking means 505. For clarity purposes, the second disengagement means 507 are only shown on FIG. 9F through FIG. 9H.

We will now describe a mode of operating the assistance device 1 according to this fifth embodiment.

In the rest state, the dispensing device 10 is in its rest position and the locking means 503 are in the locked position (see FIG. 9A). As indicated previously, in the locked position, the studs 509 of the locking means 503 are each opposite a top of the proximal end of the applicator 502. The tops of the applicator 502 come up against the tops of the studs 509 when the user tries to use the dispensing device 10 without having unlocked it, which prevents the activation of the dispensing device 10. When the user wants to use the dispensing device 10, he places his finger on the fingerprint reader 22 to unlock it in the same way as for the previous embodiments. When the user's fingerprint has been authenticated, the electronic control means via the electronic components present in the socket 20 instruct the actuator 504 to switch the pin 534 from its rest position in the base 524 to its activation position in which it projects outside the base 524. As it comes out, the pin 534 moves the protuberance 519 of the locking means 503, and therefore the locking means 503 themselves, from their locked position to their unlocked position (see FIG. 9A and FIG. 9B). The lugs 515 of the blocking means 505 have a ramp facing towards the studs 509. Each of the studs 509 of the locking means 503 moves along one of the respective ramps of the lugs 515 during the rotation of the locking means 503. As the studs 509 move along their respective ramps, the blocking means 505 are elastically deformed. When the end of each ramp of the lugs 515 has been reached, the blocking means 505 return to their initial conformation such that the studs 509 and the lugs 515 are against each other such that the blocking means 505 prevent the locking means 503 from moving towards the locked position under the action of the torsion spring (see FIG. 9B). The locking means 503 are thus blocked in the unlocked position.

In the unlocked position, the tops of the proximal end of the applicator 502 are no longer opposite the tops of the studs 509 of the locking means 503. Thus, these tops of the proximal end of the applicator 502 are no longer against the tops of the studs 509 when a user tries to activate the dispensing device 10. The dispensing device 10 can therefore be activated.

Two options are then possible: the user either activates the dispensing device 10, or does not.

If the user activates the dispensing device 10, the user moves the actuator 502 in the proximal direction, i.e. downwards in reference to FIG. 8 through FIG. 9H. During the activation, the applicator 502 is moved towards the activation position of the dispensing device 10. After a predetermined stroke, the first disengagement means 506 reach the lug 515 of the blocking means 505 and apply a force so as to elastically deform the blocking means 505 (see FIG. 9C and FIG. 9D). When the studs 509 and the lugs 515 are no longer against each other, the torsion spring of the returns means rotates the locking means 503 up to their locked position (see FIG. 9E). We are therefore back to the initial situation, i.e. the locking means 503 are in the locked position and the dispensing device 10 is in the rest position.

If the user does not activate the dispensing device 10, either because unlocking occurred accidentally, or because the user finally does not want to obtain a dose of product, it must be possible to relock the dispensing device 10, in particular to prevent an unauthorised person from using it, without however activating the dispensing device 10, in particular to avoid wasting product. To do this, a button for example and electronic control means present in the socket 20 are used to trigger the flow of an electric current in the second disengagement means 507 formed by the muscle wires (see FIG. 9F). By contracting, these muscle wires 507 elastically deform the blocking means 505 towards the outside of the assistance device 1 so as to disengage the studs 509 from the lugs 515 (see FIG. 9G). Thus, the return means rotate the locking means 503 up to the locked position (see FIG. 9H) without having to activate the dispensing device 10. As indicated previously, as an alternative or in addition, the disengagement of the locking means 503 could be controlled differently.

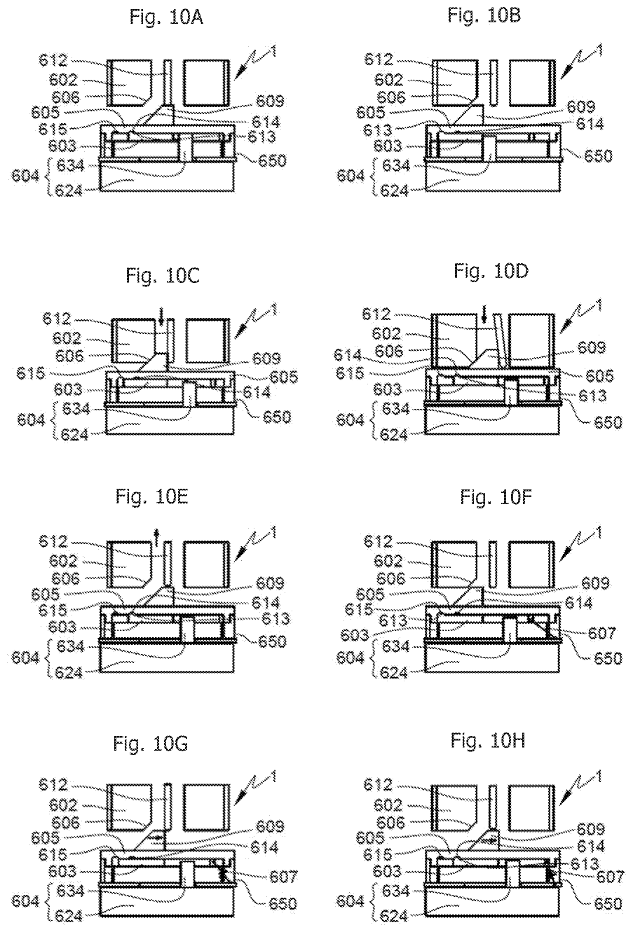

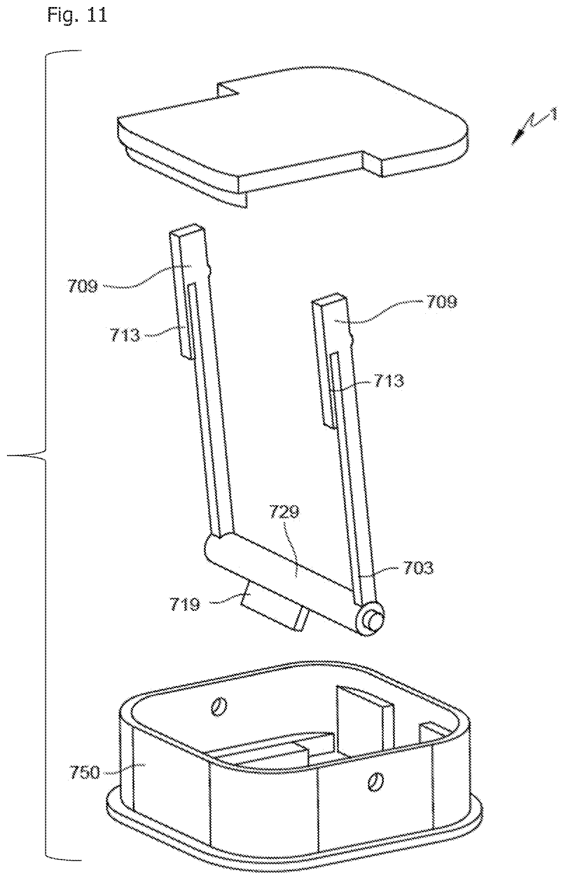

FIG. 10A through FIG. 10H show an assistance device 1 for the use of a product dispensing device 10 according to a sixth embodiment of the invention.

In this embodiment, the locking means 603 have the general shape of a flat parallelepiped and comprise studs 609 which extend perpendicularly upwards from an upper wall of the locking means 603. The studs 609 also have the general shape of a parallelepiped having one edge which has been chamfered to form a ramp. In the present case, there are two studs 609 which extend on two opposite sides of the locking means 603. There could be a different number of studs 609, for example between two and twenty, preferably between two and eight, or even between two and six. The locking means 603 are mounted on the assistance device 1 so as to be movable in horizontal translation. The locking means 603 comprise two longitudinal slots delimiting a blade having a bead-shaped lug 613 at a free end.

The assistance device 1 comprises a casing 650 capable of containing the locking means 603. The casing 650 comprises an upper wall forming blocking means 605. An inner face of the upper wall 605 has first and second cavities 614, 615 each capable of receiving the lug 613. The first cavity 614 is configured to receive the lug 613 when the locking means 603 are in the locked position and thus block the locking means 603 in this position. The second cavity 615 is configured to receive the lug 613 when the locking means 603 are in the unlocked position and thus block the locking means 603 in this unlocked position.

In this embodiment, the actuator 604 comprises a base 624 and a pin 634 movable in horizontal translation relative to the base 624 between a rest position (see FIG. 10A) and an activation position (see FIG. 10B). The pin 634 forms a projection from an upper wall of the base 624 and has a main longitudinal axis which is perpendicular to the plane containing the upper wall of the base 624 from which it extends. The pin 634 extends inside the locking means 603 through an opening in their lower wall. The pin 634 is then capable of moving the locking means 603 in horizontal translation and thus switch them from the locked position to the unlocked position.

The applicator 602 comprises two slots each on a lateral wall of the applicator 602, these two walls being opposite each other in the present case. There is a blade 612 in each slot. The slot is delimited by two proximal ends of the applicator 602, one of these ends being chamfered and thus forming the first disengagement means 606. When the locking means 603 are blocked in the unlocked position as shown on FIG. 10B, these first disengagement means 606 are capable of applying a force on the studs 609 of the locking means 603 so as to disengage the lugs 613 of the locking means 603 from the second cavity 615 of the blocking means 605 (see FIG. 10C).

The locking means 603 are movable in horizontal translation between a locked position (see FIG. 10A) in which the tops of the studs 609 are each opposite the top of one of the respective blades 612 of the applicator 602 and an unlocked position (see FIG. 10B) in which the tops of the studs 609 are not opposite the top of the blades 612 of the applicator 602 and where the chamfer of the first disengagement means 606 is closer to the ramp of the studs 609 than in the locked position.

The second disengagement means 607 comprise in this case a muscle wire having one end attached to the casing 650 and the other end attached to the locking means 603. Thus, by contracting under the effect of an electric current, the muscle wire 607 is capable of moving the locking means 603 in horizontal translation towards the locked position. The force developed by the muscle wire 607 is greater than the friction forces holding the lug 613 in the second cavity 615 of the blocking means 605. For clarity purposes, the second disengagement means 607 are only shown on FIG. 10F through FIG. 10H.

We will now describe a mode of operating the assistance device 1 according to this sixth embodiment.

In the rest state, the dispensing device 10 is in its rest position and the locking means 603 are in the locked position (see FIG. 10A). As indicated previously, in the locked position, the studs 609 of the locking means 603 are opposite the tops of the blades 612 of the applicator 602. These blades 612 come up against the tops of the studs 609 when the user tries to use the dispensing device 10 without having unlocked it, which prevents the activation of the dispensing device 10. When the user wants to use the dispensing device 10, he places his finger on the fingerprint reader 22 to unlock it in the same way as for the previous embodiments. When the user's fingerprint has been authenticated, the electronic control means via the electronic components present in the socket 20 instruct the actuator 604 to move the pin 634 in horizontal translation from its rest position to its activation position. During this translation, the pin 634, which is against an inner wall of the locking means 603, moves them in horizontal translation from their locked position to their unlocked position (see FIG. 10A and FIG. 10B). During this translation, the force applied by the pin 634 is sufficient to make the lug 613 come out of the first cavity 614 of the blocking means 605. At the end of the translation, the lug 613 engages in the second cavity 615 of the blocking means 605 such that the latter block the locking means 603 in the unlocked position (see FIG. 10B).

In the unlocked position, the tops of the blades 612 of the applicator 602 are no longer opposite the tops of the studs 609 of the locking means 603. Thus, these blades 612 are no longer against the tops of the studs 609 when a user tries to activate the dispensing device 10. The dispensing device 10 can therefore be activated (see FIG. 10B).

Two options are then possible: the user either activates the dispensing device 10, or does not.

If the user activates the dispensing device 10, the user moves the actuator 602 in the proximal direction, i.e. downwards in reference to FIG. 10A through FIG. 10H. During the activation, the applicator 602 is moved towards the activation position of the dispensing device 10. During this downward movement, the first disengagement means 606 move along the ramp of the studs 609 with sufficient force to make the lug 613 of the locking means 603 come out of the second cavity 615 of the blocking means 605. Furthermore, as the first disengagement means 606 move along the ramp of the studs 609, they move the locking means 603 in horizontal translation towards the locked position of the locking means 603 (see FIG. 100 and FIG. 10D). Thus, in this embodiment, the first disengagement means 606 also form the means for returning the locking means 603 to the locked position.

The applicator 602 is in particular allowed to move down thanks to the flexibility of the blades 612 which deform elastically under the effect of the force transmitted by the studs 609 when the locking means 603 return to the locked position (see FIG. 10D).

The applicator 602 then moves back up and the blades 612 return to their initial conformations. We are therefore back to the initial situation, i.e. the locking means 603 are in the locked position and the dispensing device 10 is in the rest position (see FIG. 10E).

If the user does not activate the dispensing device 10, either because unlocking occurred accidentally, or because the user finally does not want to obtain a dose of product, it must be possible to relock the dispensing device 10, in particular to prevent an unauthorised person from using it, without however activating the dispensing device 10, in particular to avoid wasting product. To do this, a button for example and electronic control means present in the socket 20 are used to trigger the flow of an electric current in the second disengagement means 607 formed by the muscle wire (see FIG. 10F). By contracting under the effect of an electric current, this muscle wire 607 moves the locking means 603 in horizontal translation towards the locked position (see FIG. 10G). The force developed by the muscle wire 607 is therefore greater than the friction forces holding the lug 613 in the second cavity 615 of the blocking means 605. Thus, the locking means 603 are moved in horizontal translation up to the locked position without having to activate the dispensing device 10. As indicated previously, as an alternative or in addition, the disengagement of the locking means 603 could be controlled differently.

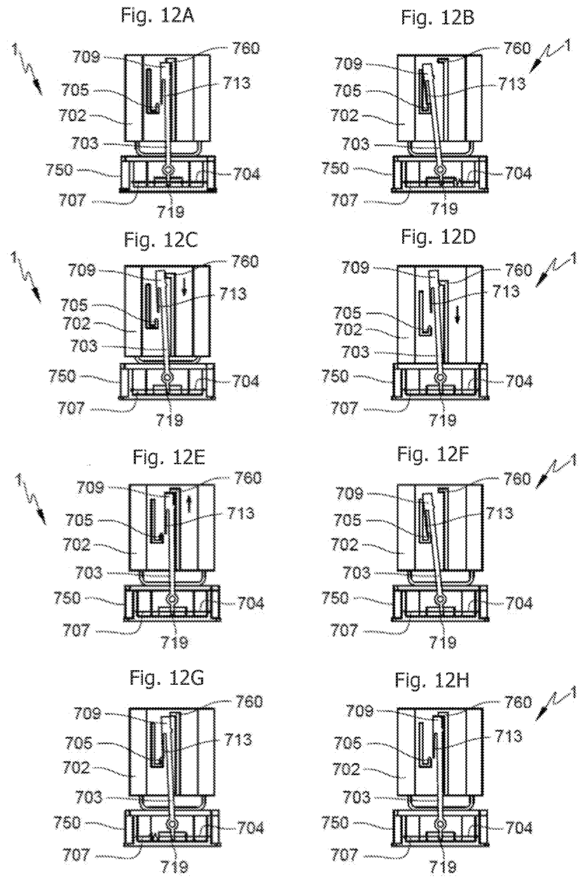

FIG. 11 through FIG. 12H show an assistance device 1 for the use of a product dispensing device 10 according to a seventh embodiment of the invention.

In this embodiment, the locking means 703 have the general shape of a circular cylinder from which studs 709 extend from each end of a cylinder 729 perpendicular to a main longitudinal axis of the cylinder 729 (see FIG. 11). The studs 709 have the general shape of a parallelepiped. In the present case, there are two studs 709. There could be a different number of studs 709, for example between two and twenty, preferably between two and eight, or even between two and six. A blade 713 extends from the free end of each stud 709 towards the other end of the stud 709 (see FIG. 11). The locking means 703 further comprise a protuberance 719 extending radially from a median portion of the cylinder. The locking means 703 are mounted on the assistance device 1 such that they can rotate about an axis perpendicular to the main longitudinal axis of the assistance device 1.

The assistance device 1 comprises a casing 750 capable of containing part of the locking means 703.

The blocking means 705 consist of two reliefs extending on an outer wall of the applicator 702 (see FIG. 12A through FIG. 12H). Each of the two reliefs 705 has:
a first straight portion oriented parallel to a main longitudinal axis of the applicator 702,
a second straight portion which follows immediately after the first portion at a proximal end of the latter and which is smaller than the latter. The second portion is oriented in a direction perpendicular to the main longitudinal axis of the applicator 702, and
a third straight portion which follows immediately after one end of the second portion and which is smaller than the latter. The third portion is oriented in a direction parallel to the main longitudinal axis of the applicator 702, and extends from the end of the second portion in the distal direction.

In the rest position, the blades 713 of the locking means 703 and the reliefs of the blocking means 705 are not engaged and are away from each other (see FIG. 12A). In the blocked position, the blades 713 of the locking means 703 are engaged with the reliefs of the blocking means 705 such that the blocking means 705 prevent the locking means 703 from rotating towards the locked position (see FIG. 12B).

The return means (not shown) configured to switch the locking means 703 from the unlocked position to the locked position comprise in this case a torsion spring. This torsion spring is compressed when the locking means 703 switch from the locked position to the unlocked position and therefore applies a force tending to return them to the locked position.

In this embodiment, the actuator comprises a muscle wire 704 having one end attached to the casing 750 and another end attached to the protuberance 719 of the locking means 703. By contracting under the effect of an electric current, this muscle wire 704 is capable of rotating the locking means 703 and switching them from the locked position (see FIG. 12A) to the unlocked position (see FIG. 12B). The actuator 702 could be any other element capable of performing this function.

The applicator 702 is configured such that when it is activated, the reliefs of the blocking means 705 move away from the blades 713 of the locking means 703 such that these two elements are no longer engaged and therefore such that the locking means 703 are no longer blocked in the unlocked position by the blocking means 705 (see FIG. 12C). Thus, the applicator 702 also forms the first disengagement means. The applicator 702 further comprises reliefs 760 on the same walls of the applicator 702 which bear the reliefs of the blocking means 705. Each of these reliefs 760 has:
a first straight portion oriented parallel to a main longitudinal axis of the applicator 702,
a second straight portion which follows immediately after the first portion at a distal end of the latter and which is smaller than the latter. The second portion is oriented in a direction perpendicular to the main longitudinal axis of the applicator 702.

The locking means 703 are mounted movable in rotation between a locked position (see FIG. 12A) in which the tops of the studs 709 are each opposite and against one of the reliefs 760 of the applicator 702 and an unlocked position (see FIG. 12B) in which the tops of the studs 709 are not opposite or against one of the reliefs 760 of the applicator 702.

The second disengagement means 707 comprise in this case a muscle wire having one end attached to the casing 750 and the other end attached to the protuberance 719 of the locking means 703. Thus, by contracting under the effect of an electric current, the muscle wire is capable of rotating the locking means 703 such that the blades 713 of the locking means 703 are no longer engaged with the reliefs of the blocking means 705.

We will now describe a mode of operating the assistance device 1 according to this seventh embodiment.

In the rest state, the dispensing device 10 is in its rest position and the locking means 703 are in the locked position (see FIG. 10A). As indicated previously, in the locked position, the studs 709 of the locking means 703 are opposite and against the reliefs 760 of the applicator 702. These reliefs 760 of the applicator 702 are against the tops of the studs 709 when the user tries to use the dispensing device 10 without having unlocked it, which prevents the activation of the dispensing device 10. When the user wants to use the dispensing device 10, he places his finger on the fingerprint reader 22 to unlock it in the same way as for the previous embodiments. When the user's fingerprint has been authenticated, the electronic control means via the electronic components present in the socket 20 cause an electric current to flow through the muscle wire 704 such that it contracts. This contraction rotates the locking means 703 from the locked position (see FIG. 12A) to the unlocked position (see FIG. 12B). The blades 713 are sufficiently elastic such that, when they come up against a part of the reliefs of the blocking means 705 under the effect of the muscle wire 704, they deform and move to the other side of the relief (see FIG. 12A and FIG. 12B). Furthermore, the blades 713 are rigid enough to remain blocked in this position. The locking means 703 are thus blocked in the unlocked position.

In the unlocked position, the reliefs 760 of the applicator 702 are no longer opposite the tops of the studs 709 of the locking means 703. Thus, these reliefs 760 no longer come up against the tops of the studs 709 when a user tries to activate the dispensing device 10. The dispensing device 10 can therefore be activated.

Two options are then possible: the user either activates the dispensing device 10, or does not.

If the user activates the dispensing device 10, the user moves the actuator 702 in the proximal direction, i.e. downwards in reference to FIG. 11 through FIG. 12H. During the activation, the applicator 702 is moved towards the activation position of the dispensing device 10. After a predetermined stroke, the reliefs of the blocking means 705 are far enough away from the blades 713 so that they are no longer engaged with them. The return means formed by the torsion spring then rotate the locking means 703 towards the locked position. However, since the applicator 702 has moved down, the locking means 703 cannot immediately return to the locked position and the studs 709 come up against the reliefs 760 (see FIG. 12D). When the applicator has completed its upward movement, the return means rotate once again the locking means 703, this time up to the locked position. We are therefore back to the initial situation, i.e. the locking means 703 are in the locked position and the dispensing device 10 is in the rest position.

If the user does not activate the dispensing device 10, either because unlocking occurred accidentally, or because the user finally does not want to obtain a dose of product, it must be possible to relock the dispensing device 10, in particular to prevent an unauthorised person from using it, without however activating the dispensing device 10, in particular to avoid wasting product. To do this, a button for example and electronic control means present in the socket 20 are used to trigger the flow of an electric current in the second disengagement means 707 formed by the muscle wire (see FIG. 12F). As it contracts, this muscle wire 707 rotates the locking means 703 towards the locked position (see FIG. 12G). The locking means 703 thus return to the locked position (see FIG. 12H) without having to activate the dispensing device 10. As indicated previously, as an alternative or in addition, the disengagement of the locking means 703 could be controlled differently.

The invention is not limited to the embodiments described and other embodiments will be clearly apparent to those skilled in the art.

1. An assistance device for assisting with the use of a product dispensing device for dispensing a product, the product dispensing being achieved by displacement or deformation of a moving part of the dispensing device between a rest position and an activation position, the assistance device comprising:
an engaging unit to the product dispensing device,
a locking unit configured to prevent the dispensing of the product,
an actuator configured to switch the locking unit from a locked position in which the lockup unit, prevents the dispensing of the product to an unlocked position in which the locking unit does not prevent the dispensing of the product,
a return unit configured to switch the locking unit from the unlocked position to the locked position, out of the activation position of the product dispensing device,
a blocking unit configured to block the locking unit in the unlocked position,
a first disengagement member for disengaging the blocking unit configured to be triggered upon the activation of the product dispensing device, and
a second disengaging the blocking member for disengaging the blocking unit configured to be triggered independently of the activation of the product dispensing device.

2. The assistance device according to claim 1, wherein the actuator comprises at least one muscle wire.

3. The assistance device according to claim 1, wherein the actuator comprises a motor.

4. The assistance device according to claim 1, wherein the second disengagement member comprises a motor.

5. The assistance device according to claim 1, wherein the second disengagement member comprises a motor.

6. The assistance device according to claim 1, wherein the second disengagement unit is configured to be activated after a predetermined time has elapsed from the switching of the locking unit to the unlocked position.

7. The assistance device according to claim 1, further comprising at least one inclination sensor for measuring an inclination of the dispensing device, wherein the second disengagement member is configured to be activated when, with the locking unit being in the unlocked position, a measurement of the inclination of the dispensing device measured by the inclination sensor reaches a predetermined threshold during a predetermined period of time.

8. The assistance device according to claim 1, wherein the locking unit is configured to switch from the unlocked position to the locked position by performing a rotational movement.

9. The assistance device according to claim 1, wherein the assistance device comprises at least one indentation configured to prevent the moving part of the dispensing device from returning to the rest position after a displacement along a predetermined stroke of the moving part of the dispensing device, the predetermined stroke being shorter than a total stroke corresponding to the stroke leading to the activation position of the dispensing device.
